(19) United States
(12) Patent Application Publication
(10) Pub. No.: US 2018/0263358 A1
(43) Pub. Date: Sep. 20, 2018

(54) AGENT AND METHOD FOR COSMETIC TREATMENT

(71) Applicant: Henkel AG & Co. KGaA, Dusseldorf (DE)

(72) Inventors: Thomas Foerster, Duesseldorf (DE); Rolf Bayersdoerfer, Hamburg (DE); Thorsten Knappe, Schenefeld (DE)

(73) Assignee: Henkel AG & Co. KGaA, Duesseldorf (DE)

(21) Appl. No.: 15/534,655

(22) PCT Filed: Nov. 2, 2015
(86) PCT No.: PCT/EP2015/075394

§ 371 Date: Jun. 9, 2017

(30) Foreign Application Priority Data
Dec. 10, 2014 (DE) 10 2014 225 434.5

(51) Int. Cl. A45D 34/04; A61Q 13/00; A61K 8/34; A61Q 5/00; A61Q 19/00; A61K 8/86; A45D 34/02

(52) U.S. Cl. CPC A45D 34/04; A61Q 13/00; A61K 8/34; A61Q 5/00; A45D 2200/057; A61K 8/86; A45D 34/02; A61K 2800/87; A45D 2200/155; A61Q 19/00

(57) ABSTRACT

A cosmetic product is provided herein. The cosmetic product includes a cosmetic preparation including, relative to the total weight thereof, from about 20 to about 99 wt % ethanol and from about 0.05 to about 20 wt % fragrance. The cosmetic product further includes a device for flash evaporation of cosmetic preparation.

### CROSS-REFERENCE TO RELATED APPLICATION

This application is a U.S. National-Stage entry under 35 U.S.C. § 371 based on International Application No. PCT/EP2015/075394, filed Nov. 2, 2015, which was published under PCT Article 21(2) and which claims priority to German Application No. 10 2014 225 434.5, filed Dec. 10, 2014, which are all hereby incorporated in their entirety by reference.

### TECHNICAL FIELD

The application relates to the technical field of treatment of human skin and human hair. The subject matter of the application is specific cosmetic formulations which are suitable for application to the skin and hair by employing a flash evaporation process. A further object of the present application is the use of said cosmetic formulations in devices for flash evaporation and methods for treating hair.

### BACKGROUND

For many people, perfuming of the skin or hair is an essential aspect of daily personal care.

In the field of perfumery, the spray application of preparations that contain fragrances is extremely important, the preparations generally being applied as pump sprays or aerosol sprays. For this, the fragrance-containing preparations are packaged in a dispensing device from which they are sprayed either by mechanical force or with the aid of a propellant through a valve. Both methods have evident drawbacks. While pump sprays typically do not lend themselves well for long lasting uniform spray application preparations, even for sprayed application of fragrance-containing preparations, aerosol sprays, which are based on the use of blowing agents or propellants having no cosmetic effect themselves, may also present a risk of injury to consumers if misused.

Against this background there is a need for alternative ways to atomize fragrance-containing preparations. Flash evaporation has proven effective as such an alternative spraying method. In this method, which is described for example in International Patent Application WO 2001/83071 A1 (Henkel), a liquid or paste-like solvent-containing composition is heated in an enclosed space to a temperature above the boiling point of the solvent, whereby overpressure is generated in the composition. When the pressure is released (throttling), the liquid evaporates and can then be atomized, by employing a suitable nozzle for example.

But while flash evaporation is generally suitable for the spray application of fragrance-containing preparations, at the same time not all hair cosmetic preparations can be atomized by employing a flash evaporation process. This is due on the one hand to the heating of the cosmetic preparation that is needed for flash evaporation, and on the other hand to the specifics of the spray mist produced by flash evaporation, for example the droplet size and droplet density produced in the spray.

### BRIEF SUMMARY

A cosmetic product is provided herein. The cosmetic product includes a cosmetic preparation including, relative to the total weight thereof, from about 20 to about 99 wt % ethanol and from about 0.05 to about 20 wt % fragrance. The cosmetic product further includes a device for flash evaporation of cosmetic preparation.

A cosmetic composition is also provided herein. The cosmetic composition includes, relative to the total weight thereof, from about 20 to about 99 wt % ethanol and from about 0.05 to about 20 wt % fragrance. The cosmetic composition is utilized as a material in a flash evaporation device.

A method for fragrancing human skin or human hair is also provided herein. The method includes applying a cosmetic composition to the hair or skin by employing a flash evaporation device. The cosmetic composition includes, relative to the total weight thereof, from about 20 to about 99 wt % ethanol and from about 0.05 to about 20 wt % fragrance.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide specific fragrance-containing preparations for perfuming the skin or hair which by their chemical and physical properties are suitable for targeted spray application by employing an apparatus for flash evaporation. The preparations should also be suitable for delivering good fragrancing following an application by employing a flash evaporation process. It has been found that, of the many known fragrance-containing preparations, alcoholic fragrance preparations lend themselves particularly to achieving this object.

A first subject matter of the present disclosure is therefore a cosmetic product comprising
a) a cosmetic preparation containing, relative to total weight,
   a1) from about 20 to about 99 wt % ethanol;
   a2) from about 0.05 to about 20 wt % fragrance;
b) a device for flash evaporation of cosmetic preparation a).

Cosmetic preparation a) is preferably liquid. Cosmetic preparation a) may be present as a solution or a dispersion, for example as an emulsion or suspension. Preferred cosmetic preparations a) are in the form of a solution or a suspension.

The cosmetic preparation as contemplated herein contains as the first essential component from about 20 to about 99 wt % ethanol a1). Preferred cosmetic products are characterized in that the proportion by weight ethanol a1) of the total weight of cosmetic preparation a) is from about 30 to about 90 wt %, preferably from about 40 to about 80 wt %. Compositions of such kind are characterized by good cosmetic effect together with good application properties.

Ethanol a1) may be used as the only solvent or in combination with one or more additional polar solvents. In order to improve the application properties of the cosmetic preparations as contemplated herein while minimizing the thermal load of any active or auxiliary substances during the flash evaporation process, it has proved advantageous to use polar solvents which have a boiling temperature (20° C., 1013 mbar) between about 50 and about 110° C., preferably between about 70 and about 105° C. Water has been found to be particularly suitable, and for this reason is used preferably as an additional polar solvent.

Preferred cosmetic preparations a) are characterized in that the proportion by weight of water and ethanol of the total weight thereof is at least about 60 wt %, preferably at least about 80 wt %, particularly preferably at least about 90 wt % and especially at least about 95 wt %.

In the cases in which cosmetic preparation a comprises ethanol and water, it is preferable if the weight ratio of ethanol to water is in the range from about 50:1 to about 1:2, preferably about 40:1 to about 1:1 and especially about 30:1 to about 10:1.

A second essential ingredient of cosmetic compositions as contemplated herein is the fragrance a2). With regard to their application properties, cosmetic preparations a) in which the proportion by weight of the fragrance relative to the total weight of cosmetic preparation a) is from about 0.1 to about 15 wt %, and particularly from about 0.5 to about 10 wt % have proven advantageous.

The term “fragrances” includes both natural and synthetic substances that can be perceived in an olfactory manner and develop a smell. Fragrance a) may be a single chemical substance, but also a mixture of substances, wherein the use of substance mixtures is preferred as contemplated herein due to the more complex scent.

In order to be perceptible, a fragrance must be volatile, and in such case molecular weight plays an important role as well as the nature of the functional groups and the structure of the chemical compound. Thus, most perfumes have molecular weights up to about 200 daltons, with molecular weights of about 300 daltons and above rather representing the exception. Due to the different volatilities of the fragrances, the smell of a perfume or scent composed of several aroma substances changes as it evaporates, wherein the olfactory impressions are divided into the “top note”, the “middle note” or “body”, and the “end note” or “dry out”. Since smell perception is also determined to a large by smell intensity, the top note of a perfume or fragrance consists of more than just the readily volatile compounds, while the end note consists mostly of less volatile i.e. solid fragrances.

In the course of application of fragrances by flash evaporation, the boiling point of the fragrances is of some importance. It has proven advantageous for the application and cosmetic effect of cosmetic preparation a) if fragrance a2) has a boiling point above about 80° C., preferably above about 120° C. and particularly above about 160° C. Preferably at least about 60 wt %, more preferably at least about 80 wt % and particularly at least about 90 wt % of fragrance a2) has a boiling point above about 80° C., preferably above about 120° C. and particularly above about 160° C.

For the reasons mentioned above, the higher-boiling or solid odorants of natural or synthetic origin are particularly preferred within the scope of the present disclosure. These substances include, for example ambrettolide, α-amyl cinnamic aldehyde, anethole, anisaldehyde, anise alcohol, anisole, methyl anthranilate, acetophenone, benzyl acetone, benzaldehyde, ethyl benzoate, benzophenone, benzyl alcohol, benzyl acetate, benzyl benzoate, benzyl formiate, benzyl valerianate, borneol, bornyl acetate, α-bromostyrene, n-decylaldehyde, n-dodecylaldehyde, eugenol, eugenol methyl ether, eucalyptol, farnesol, fenchone, fenchyl acetate, geranyl acetate, geranyl formiate, heliotropin, heptin carboxylic acid methylester, heptaldehyde, hydroquinone dimethyl ether, hydroxycinnamaldehyde, hydroxycinnamyl alcohol, indole, iron, isoeugenol, isoeugenol methyl ether, isosafrole, jasmone, camphor, carvacrol, carvone, p-cresol methylether, coumarin, p-methoxyacetophenone, methyl-n-amylketone, methyl anthranilate methyl ester, p-methylacetophenone, methylchavikol, p-methylquinoline, methyl-β-naphthyl ketone, methyl-n-nonyl acetaldehyde, methyl-n-nonyl ketone, muskone, β-naphthol ethylether, β-naphthol methylether, nerol, nitrobenzene, n-nonylaldehyde, nonyl alcohol, n-octylaldehyde, p-oxy-acetophenone, pentadecanolide, β-phenylethyl alcohol, phenyl acetaldehyde-dimethyacetal, phenylacetic acid, pulegone, safrole, salicylic acid isoamyl ester, methyl salicylate, hexyl salicylate, cyclohexyl salicylate, santalol, skatole, terpineol, thymene, thymol, γ-undelactone, vanillin, veratraldehyde, cinnamic aldehyde, cinnamyl alcohol, cinnamic acid, ethyl cinnamate and benzyl cinnamate.

As an alternative to the higher-boiling or solid odoriferous substances or in combination with these, the adherent odorants are also preferably used as perfumes a2). The group of these fragrances includes among others the essential oils such as angelica root oil, anise oil, arnica flower oil, basil oil, bay oil, bergamot oil, champaca flower oil, silver fir oil, silver fir cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, neroli oil, niaouli oil, olibanum oil, orange oil, oregano oil, palmarosa oil, patchouli oil, peru balsam oil, petitgrain oil, pepper oil, peppermint oil, allspice oil, pine oil, rose oil, rosemary oil, sandalwood oil, celery oil, lavender spike oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil.

Besides cosmetic preparation a), the cosmetic products as contemplated herein also comprise an apparatus for flash evaporation. Within the scope of the present application, the term “flash evaporation” means the generation of vapor by lowering the pressure in an enclosed space that is filled with a liquid and under overpressure (compared with the environment). A corresponding overpressure may be produced for example by heating a quantity of the cosmetic preparation a) to a temperature T1i in an enclosed space. In the enclosed space, the liquid has a pressure saturation pressure p1 at the given temperature T1. If the enclosed space is opened, by employing a valve for example, and transformed into a relaxation space which is not under overpressure, where pressure po<p1, the pressure in the previously enclosed space falls, and as the new pressure level is established through the space the cosmetic preparation a) and the solvent contained in the cosmetic preparation or parts thereof are vaporized. The resulting vapor or spray mist may be used for the application of specific cosmetic preparations.

Thus, if cosmetic preparation a) is heated in an enclosed space beginning from standard conditions (To=25° C., po=1.000 bar), apart from increased temperature this results in increased pressure in cosmetic preparation a). This increased pressure can be relieved in a relaxation space to a pressure po, ambient air pressure for example (po=1,000 bar), thereby causing at least partial evaporation of cosmetic preparation a).

Cosmetic preparation a) may be relaxed directly in the space in which it was previously heated. Alternatively, the heated and pressurized cosmetic preparation a) may also be transported into a second space after heating, in which the pressure is then relieved.

In other words, flash evaporation is a method in which cosmetic preparation a) is heated in an enclosed container by a heater to temperatures above the ambient temperature, wherein a pressure above ambient pressure is created inside the container and the heated cosmetic preparation a) under increased pressure is then depressurized from the container into the environment.

An apparatus for flash evaporation is therefore a device comprising a container and a heater that is configured such that a cosmetic composition a) may be heated by employing the heater in the closed container to temperatures above the ambient temperature in such manner that a pressure above ambient pressure is created inside the container and the cosmetic preparation a) which is heated and under increased pressure may be released from the container into the surrounding atmosphere.

Simultaneously with or after the pressure relief, cosmetic preparation a) may be forwarded to a nozzle such that it is possible to influence properties of the vapor or spray generated by the flash evaporation, for example, particularly the droplet size or droplet density as well as the spraying range and the shape of the spray cone. The use of nozzles, preferably atomizing nozzles, is therefore, preferred. The specific nozzle type or nozzle design is defined specifically for the respective spray mist characteristics.

In summary, a preferred apparatus for flash evaporation includes
b1) a container b1) which can be opened and closed by employing a valve and which defines the enclosed inner space in which the cosmetic preparation may be accommodated,
b2) a heater b2), which makes it possible to heat a cosmetic preparation inside container b1).

Use of an additional nozzle b3) which enables atomisation of the cosmetic preparation a) as it escapes from the container is particularly preferred. Instead of a valve, a similarly acting closing element which can block or release an associated opening in the container by a corresponding change in position, may also be used.

A preferred object of the present disclosure is a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), wherein the apparatus for flash evaporation comprises a container b1) and a heater b2) and is configured such that
   the cosmetic preparation a) can be accommodated in the interior of container b1),
   the interior space of container b1) which is at least partly filled with cosmetic preparation a) may be closed off,
   cosmetic preparation a) may be heated in the closed interior of container b1) by heater b2), increasing the pressure therein.

A particularly preferred object of the present disclosure is therefore a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle B3) which enables atomisation of the cosmetic preparation a).

In other words, a particularly preferred object of the present disclosure is a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), wherein the apparatus for flash evaporation comprises a container b1) and a heater b2) and is configured such that
   the cosmetic preparation a) can be accommodated in the interior of container b1),
   the interior space of container b1) which is at least partly filled with cosmetic preparation a) may be closed off,
   cosmetic preparation a) may be heated in the closed interior of container b1) by heater b2), increasing the pressure therein,
   the heated cosmetic preparation a) may be relaxed out of the interior of container b1) into the surrounding atmosphere.

The container b1) in which the cosmetic preparation is heated is designed in a way that makes it possible to close this container off from the environment completely while cosmetic preparation a) is being heated, and after the preparation has been heated to open the container to allow flash evaporation of cosmetic preparation a). This may be ensured for example by a flow controlling component, particularly a valve.

The container b1) in which the cosmetic preparation is heated preferably communicates with another container, from which the quantity of cosmetic preparation intended to undergo flash evaporation prior is transferred to container b1) prior to heating. Access between this reservoir and container b1) is openable and closable by employing an appropriate device, such as a valve. This further container is preferably designed as a reservoir, that is to say it preferably contains several times, for example more than about ten times, preferably more than about fifty times the quantity of the cosmetic preparation required for an evaporation operation. In other words, the volume of the further container/reservoir is preferably several times, for example more than about ten times, preferably more than about twenty times and in particular more than about fifty times greater than the volume of the container b1).

A further, particularly preferred object of the present disclosure is therefore a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle b3) which enables atomisation of the cosmetic preparation a)
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least about ten times greater, preferably at least about twenty times greater and particularly at least about fifty times greater than that of container b1).

The reservoir is not a pressure vessel, and the cosmetic composition in the reservoir is not under pressure, in other words the pressure inside the reservoir is equal to the ambient pressure (also called air pressure or atmospheric pressure). Accordingly, cosmetic products of such kind do not comprise a propellant, for example. The cosmetic product also has no pumping device that is capable of releasing or spraying the cosmetic preparation into the environment without the action of the apparatus for flash evaporation.

A particularly preferred object of the present disclosure is therefore a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle b3) which enables atomisation of the cosmetic preparation a)
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least about ten times greater, preferably at least about twenty times greater and particularly at least about fifty times greater than that of container b1);
   the pressure inside the reservoir is equal to the ambient pressure.

A particularly preferred object of the present disclosure is therefore a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle b3) which enables atomisation of the cosmetic preparation a)
c) a reservoir for cosmetic preparation a), which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least about ten times greater, preferably at least about fifty times greater than that of container b1);
   the pressure inside the reservoir is equal to the ambient pressure and the cosmetic product contains no propellant.

Moreover, cosmetic products are preferred which comprise
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle b3) which enables atomisation of the cosmetic preparation a)
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least about ten times greater, preferably at least about fitly times greater than that of container b1);
   the pressure inside the reservoir is equal to the ambient pressure,
      wherein the cosmetic product does not have a pump device designed to release or spray cosmetic preparation a) without the action of the apparatus for flash evaporation.

In summary, a particularly preferred object of the present disclosure is therefore a cosmetic product comprising
a) a cosmetic preparation comprising, relative to the total weight thereof,
   a1) from about 20 to about 99 wt % ethanol;
   a2) from about 0.05 to about 20 wt % fragrance;
b) a device for flash evaporation of cosmetic preparation a), comprising
   b1) a container b1) which may be closed and opened by employing a valve
   b2) a heater b2), which makes it possible to heat a cosmetic preparation inside container b1)
   b3) a nozzle b3) which enables atomisation of the cosmetic preparation a)
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least about ten times greater, preferably at least about fifty times greater than that of container b1);
   the pressure inside the reservoir is equal to the ambient pressure and the cosmetic product contains no propellant,
      wherein the cosmetic product does not have a pump device designed to release or spray cosmetic preparation a) without the action of the apparatus for flash evaporation.

Apart from the two components a1) and a2) described previously, the cosmetic preparations a) as contemplated herein may contain further active or auxiliary substances, wherein those active or auxiliary substances are particularly preferred which improve the manufacturability, ease of application and/or cosmetic effect of the inventive cosmetic preparations.

To improve manufacturability, ease of application and cosmetic effect, cosmetic preparation a) preferably contains a nonionic surfactant a3), wherein particularly preferred cosmetic preparations a) are characterized in that they contain from about 0.02 to about 4.0 wt %, preferably from about 0.05 to about 2.0 wt % and particularly from about 0.1 to about 1.0 wt % non-ionic surfactant a3) relative to the total weight thereof.

Preferred non-ionic surfactants are PEG derivatives of hydrogenated castor oil, available for example under the name PEG hydrogenated castor oil, such as PEG-30 Hydrogenated Castor Oil, PEG-33 Hydrogenated Castor Oil, PEG-35 Hydrogenated Castor Oil, PEG 36 Hydrogenated Castor Oil, PEG-40 Hydrogenated Castor Oil or PEG-60 Hydrogenated Castor Oil. As contemplated herein, particularly preferred non-ionic surfactants are selected from the group of PEG derivatives of hydrogenated castor oil, particularly preferably from the group of PEG-40 Hydrogenated Castor Oil, and PEG-60 Hydrogenated Castor Oil, especially PEG-40 Hydrogenated Castor Oil.

To improve their aroma profile, preferred cosmetic preparations a) comprise a film-forming polymer a4). Particularly suitable for use as film-forming polymers a4) are both permanently and temporarily cationic, anionic, non-ionic or amphoteric polymers. These film-forming polymers may be of synthetic or natural origin. Preferred cosmetic preparations a) contain from 0.1 to 20 wt %, preferably from 0.5 to 15 wt % and particularly from 1.0 to 10 wt % of a film-forming polymer a4) relative to the total weight thereof.

Examples of usual film-forming polymers a3) are Acrylamide/Ammonium Acrylate copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates copolymer, Acrylates/Acetoacetoxyethyl Methacrylate Copolymer, Acrylates/Acrylamide Copolymer, Acrylates/Ammonium Methacrylate copolymer, Acrylates/t-Butylacrylamide copolymer, Acrylates copolymer, Acrylates/C1-2 Succinates/Hydroxyacrylates copolymer, Acrylates/Lauryl Acrylate/Stearyl Acrylate/Ethylamine Oxide Methacrylate copolymer, Acrylates/Octylacrylamide copolymer, Acrylates/Octylacrylamide/Diphenyl Amodimethicone copolymer, Acrylate/Stearyl Acrylate/Ethylamine Oxide Methacrylate copolymer, Acrylates/VA copolymer, Acrylates/VP copolymer, Adipic Acid/Diethylenetriamine copolymer, Adipic Acid/Dimethylaminohydroxypropyl Diethylenetriamine copolymer, Adipic Acid/Epoxypropyl Diethylenetriamine copolymer, Adipic Acid/Isophthalic Acid/Neopentyl Glycol/Trimethylolpropane copolymer, Allyl Stearate/VA copolymer, Aminoethylacrylate Phosphate/Acrylates copolymer, Aminoethylpropanediol-Acrylates/Acrylamide copolymer, Aminoethylpropanediol-AMPD-Acrylates/Diacetoneacrylamide copolymer, Ammonium VA/Acrylates copolymer, AMPD-Acrylates/Diacetoneacrylamide copolymer, AMP-Acrylates/Allyl Methacrylate copolymer, AMP Acrylates/C1-18 Alkyl Acrylates/C1-8 Alkyl Acrylamide copolymer, AMP Acrylates/Diacetoneacrylamide copolymer, AMP-Acrylates/Dimethylaminoethylmethacrylate copolymer, Bacillus/Rice Bran Extract/Soybean Extract Ferment Filtrates, Bis-Butyloxyamodimethicone/PEG-60 copolymer, Butyl Acrylate/Ethylhexyl Methacrylate copolymer, Butyl Acrylate/Hydroxypropyl Dimethicone Acrylate copolymer, Butylated PVP, Butyl Ester of Ethylene/MA copolymer, Butyl Ester of PVM/MA copolymer, Calcium/Sodium PVM/MA copolymer, Corn Starch/Acrylamide/Sodium Acrylate copolymer, Diethylene Glycolamine/Epichlorohydrin/Piperazine copolymer, Dimethicone Crosspolymer, Diphenyl Amodimethicone, Ethyl Ester of PVM/MA copolymer, Hydrolyzed Wheat Protein/PVP Crosspolymer, Isobutylene/Ethylmaleimide/Hydroxyethylmaleimide copolymer, Isobutylene/MA copolymer Isobutylmethacrylate/Bis-Hydroxyropyl Dimethicone Acrylate copolymer, Isopropyl Ester of PVM/MA copolymer, Lauryl Acrylate Crosspolymer, Lauryl Methacrylate/Glycol Dimethacrylate Crosspolymer, MEA-sulfites, Methacrylic Acid/Sodium Acrylamidomethyl Propane Sulfonate copolymer, Methacryloyl Ethyl Betaine/Acrylates copolymer, Octylacrylamide/Acrylates/Butylaminoethyl Methacrylate copolymer, PEG/PPG-25/25 Dimethicone/Acrylates copolymer, PEG-8/SMDI copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene,

Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12 , Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA copolymer, Potassium Ethyl Ester of PVM/MA copolymer, PPG-70 Polyglyceryl-10 Ether, PPG-12/SMDI copolymer, PPG-51/SMDI copolymer, PPG-10 Sorbitol, PVM/MA copolymer, PVP, PVP/VA/Itaconic Acid copolymer, PVP/VA/Vinyl Propionate copolymer, Rhizobian Gum, Rosin Acrylate, Shellac, Sodium Butyl Ester of PVM/MA copolymer, Sodium Ethyl Ester of PVM/MA copolymer, Sodium Polyacrylate, Sterculia Urens Gum, Terephthalic Acid/Isophthalic Acid/Sodium Isophthalic Acid Sulfonate/Glycol copolymer, Trimethylolpropane Triacrylate Trimethylsiloxysilyl-carbamoyl Pullulan, VA/Crotonates copolymer, VA/Crotonates/Methacryloxybenzophenone-1 copolymer, VA/Crotonates/Vinyl Neodecanoate copolymer, VA/Crotonates/Vinyl Propionate copolymer, VA/DBM copolymer, VA/Vinyl Butyl Benzoate/Crotonates copolymer, Vinylamine/Vinyl Alcohol copolymer, Vinyl Caprolactam/VP/Dimethylaminoethyl Methacrylate copolymer, VP/Acrylates/Lauryl Methacrylate copolymer, VP/Dimethylaminoethylmethacrylate copolymer, VP/DMAPA Acrylates copolymer, VP/Hexadecene copolymer, VP/VA copolymer, VP/Vinyl Caprolactam/DMAPA Acrylates copolymer, Yeast Palmitate and Styrene/VP copolymer.

Additional care products are also particularly worthy of note as suitable active or auxiliary substances.

The substance may contain for example at least one protein hydrolysate and/or a derivative thereof as the care product. Protein hydrolysates are product mixtures obtained by acid-, base- or enzyme-catalyzed breakdown of proteins. For the purposes of the present disclosure, the term protein hydrolysate is understood to include total hydrolysates as well as individual amino acids and their derivatives and mixtures of different amino acids. The molecular weight of the protein hydrolysates that are usable as contemplated herein is between about 75, the molecular weight of glycine, and about 200,000. The molecular weight is preferably between about 75 and about 50,000 and most particularly preferably between about 75 and about 20,000 daltons.

The inventive substance may further contain at least one vitamin, one provitamin, one vitamin precursor and/or a derivative thereof as a care product. In this context, vitamins, provitamins and vitamin precursors that are usually assigned to groups A, B, C, E, F and H are preferred for the purposes of the disclosure.

Additional care products are panthenol, caffeine, nicotinamide and sorbitol.

The inventive substances may further contain at least one plant extract, but also mono- or oligosaccharides and/or lipids as a care product.

The compositions of some cosmetic preparations that are particularly preferred as contemplated herein are listed in the following tables (values in wt % relative to the total weight of the cosmetic composition unless otherwise specified). With respect to further preferred variants of these particularly preferred compositions, the earlier notes on the inventive cosmetic preparations a) still apply mutatis mutandis.

| | Formula 1 | Formula 2 | Formula 3 | Formula 4 | Formula 5 |
|---|---|---|---|---|---|
| Ethanol | 20 to 99 | 30 to 90 | 40 to 88 | 54 | 84 |
| Fragrance a2) | 0.05 to 20 | 0.1 to 15 | 0.5 to 10 | 0.7 | 2.3 |
| Optional | add 100 | add 100 | add 100 | add 100 | add 100 |
| additives | | | | | |
| | Formula 6 | Formula 7 | Formula 8 | Formula 9 | Formula 10 |
| Ethanol | 80 to 99 | 90 to 99 | 95 to 99 | 98 | 96 |
| Fragrance a2) | 0.05 to 20 | 0.1 to 15 | 0.5 to 10 | 0.7 | 2.3 |
| Optional | add 100 | add 100 | add 100 | add 100 | add 100 |
| additives | | | | | |
| | Formula 11 | Formula 12 | Formula 13 | Formula 14 | Formula 15 |
| Ethanol | 20 to 99 | 30 to 90 | 40 to 88 | 54 | 84 |
| Fragrance a2) | 0.05 to 20 | 0.1 to 15 | 0.5 to 10 | 0.7 | 2.3 |
| Nonionic | 0.02 to 4.0 | 0.05 to 2.0 | 0.1 to 1.0 | 0.4 | 0.1 |
| surfactant a3) | | | | | |
| Optional | add 100 | add 100 | add 100 | add 100 | add 100 |
| additives | | | | | |
| | Formula 16 | Formula 17 | Formula 18 | Formula 19 | Formula 20 |
| Ethanol and | 80 to 99 | 90 to 99 | 95 to 99 | 98 | 96 |
| water | | | | | |
| Fragrance a2) | 0.05 to 20 | 0.1 to 15 | 0.5 to 10 | 0.7 | 2.3 |
| Nonionic | 0.02 to 4.0 | 0.05 to 2.0 | 0.1 to 1.0 | 0.4 | 0.1 |
| surfactant a3) | | | | | |
| Optional | add 100 | add 100 | add 100 | add 100 | add 100 |
| additives | | | | | |
| | Formula 21 | Formula 22 | Formula 23 | Formula 24 | Formula 25 |
| Ethanol | 20 to 99 | 30 to 90 | 40 to 88 | 54 | 84 |
| Fragrance a2) | 0.05 to 20 | 0.1 to 15 | 0.5 to 10 | 0.7 | 2.3 |
| with boiling | | | | | |
| point | | | | | |
| >120° C. | | | | | |
| Optional | add 100 | add 100 | add 100 | add 100 | add 100 |
| additives | | | | | |
| | Formula 26 | Formula 27 | Formula 28 | Formula 29 | Formula 30 |
| Ethanol and | 80 to 99 | 90 to 99 | 95 to 99 | 98 | 96 |
| water | | | | | |
| Fragrance a2) | 0.05 to 20 | 0.1 to 15 | 0.5 to 10 | 0.7 | 2.3 |
| with boiling | | | | | |
| point | | | | | |
| >120° C. | | | | | |
| Optional | add 100 | add 100 | add 100 | add 100 | add 100 |
| additives | | | | | |
| | Formula 31 | Formula 32 | Formula 33 | Formula 34 | Formula 35 |
| Ethanol | 20 to 99 | 30 to 90 | 40 to 88 | 54 | 84 |
| Fragrance a2) | 0.05 to 20 | 0.1 to 15 | 0.5 to 10 | 0.7 | 2.3 |
| with boiling | | | | | |
| point | | | | | |
| >120° C. | | | | | |
| Nonionic | 0.02 to 4.0 | 0.05 to 2.0 | 0.1 to 1.0 | 0.4 | 0.1 |
| surfactant a3) | | | | | |
| Optional | add 100 | add 100 | add 100 | add 100 | add 100 |
| additives | | | | | |
| | Formula 36 | Formula 37 | Formula 38 | Formula 39 | Formula 40 |
| Ethanol and | 80 to 99 | 90 to 99 | 95 to 99 | 98 | 96 |
| water | | | | | |
| Fragrance a2) | 0.05 to 20 | 0.1 to 15 | 0.5 to 10 | 0.7 | 2.3 |
| with boiling | | | | | |
| point | | | | | |
| >120° C. | | | | | |
| Nonionic | 0.02 to 4.0 | 0.05 to 2.0 | 0.1 to 1.0 | 0.4 | 0.1 |
| surfactant a3) | | | | | |
| Optional | add 100 | add 100 | add 100 | add 100 | add 100 |
| additives | | | | | |

Besides the components a1) to a3) described above, very particularly preferred cosmetic preparations contain only small quantities of other active ingredients and excipients. Cosmetic preparations characterized in that the proportion by weight of components a1), a2), plus a3) if included, is at least about 80 wt %, preferably at least about 90 wt %, particularly preferably at least about 93 wt % and most particularly at least about 97 wt % of the total weight of the cosmetic preparation, are particularly preferred because they are simple to manufacture and have good cosmetic effect. Very particularly preferred cosmetic preparations consist of at least about 80 wt %, preferably at least about 90 wt % and particularly at least about 97 wt % of components a1) and a2) relative to their total weight.

Besides the components a1) to a5) described above, very particularly preferred cosmetic preparations contain only small quantities of other active ingredients and excipients. Cosmetic preparations characterized in that the proportion by weight of components a1), a2), plus a3) and/or a4) and/or a5) if included, is at least about 80 wt %, preferably at least about 90 wt %, particularly preferably at least about 93 wt % and most particularly at least about 97 wt % of the total weight of the cosmetic preparation, are particularly preferred because they are simple to manufacture and have good cosmetic effect. Very particularly preferred cosmetic preparations consist of at least about 80 wt %, preferably at least about 87 wt % and particularly at least about 95 wt % of components a1), a2) and a3) relative to their total weight.

As was noted in the introduction, the cosmetic preparations a) as contemplated herein lend themselves particularly well to application by employing an apparatus for flash evaporation. further object of the present application is therefore the use of a cosmetic formulation a) comprising, relative to the total weight thereof,
a1) from about 20 to about 99 wt % ethanol;
a2) from about 0.05 to about 20 wt % fragrance;
   as process materials in a flash evaporation device.

Another object of the present application is the use of a product as contemplated herein for spraying on human skin or human hair, with a cosmetic preparation a) and/or for perfuming human skin or human hair.

A method for perfuming human skin or human hair in which the keratin-containing fibres are exposed by employing a flash evaporation device to a cosmetic preparation a) comprising
a1) from about 20 to about 99 wt % ethanol;
a2) from about 0.05 to about 20 wt % fragrance
   relative to the total weight thereof, is a further object of the present application. Cosmetic composition a) is preferably converted into a spray mist by employing the apparatus for flash evaporation, which mist is subsequently sprayed over the keratin-containing fibres.

In order to achieve adequate spraying effect, cosmetic preparation a) is preferably heated to temperatures above the boiling point of the polar solvent or solvent mixture contained in cosmetic preparation a).

If the polar solvent is water or solvent mixtures with a water content greater than about 50 wt % (relative to the total weight of the solvent mixture), the cosmetic preparation is preferably heated to temperatures above about 100° C., more preferably to temperatures between about 100° C. and about 240° C., particularly preferably to temperatures between about 140° C. and about 160° C.

In the cases in which the polar solvent is water or solvent mixtures with a water content greater than about 50 wt % (relative to the total weight of the solvent mixture), the overpressure achieved by heating cosmetic preparation a) is preferably between about 1.1 and about 8 bar, preferably between about 1.2 and about 4 bar.

A preferred object of the application is a method for changing the color of keratin fibers, particularly human hair, in which the keratin-containing fibres are sprayed with a cosmetic preparation a) containing
a1) from about 20 to about 99 wt % ethanol;
a2) from about 0.05 to about 20 wt % fragrance
   relative to the total weight thereof, by employing an apparatus for flash evaporation, wherein
   a partial quantity of the cosmetic preparation a) present in a reservoir, inside which a pressure corresponding to ambient pressure prevails, is transferred from said reservoir to a container b1);
   the flow from the reservoir to container b1) is then interrupted by a flow control component, such that the flow of cosmetic preparation a) from the reservoir into container b1) may be interrupted;
   then, the cosmetic preparation a) in container b1) which is closed off from the surrounding atmosphere is heated by a heater, so that the pressure inside container b1) increases to values above ambient pressure, preferably to values between about 1.1 and about 8 bar, particularly to values between about 1.2 and about 4 bar;
   then, the container b1) under a pressure above ambient pressure opens in such manner as to allow at least a partial quantity, preferably at least about 50 wt %, more preferably at least about 80 wt % and particularly at least about 90 wt % of the cosmetic preparation inside container b1) to escape from the container b1) into the environment, at the same time reducing the pressure that prevails in container b1) at the time container b1) is opened.

The relaxation of the cosmetic preparation a) into the surrounding atmosphere preferably causes the formation of a spray mist of cosmetic preparation a).

The cosmetic preparation a) which is able to relax out of container b1) is preferably directed at keratin fibers, particularly human hair.

Methods during which the cosmetic preparation that relaxes out of container b1) is directed through a nozzle before it reaches the keratin fibers are particularly preferred.

The previous notes and explanations regarding the inventive cosmetic preparations a) and apparatus for flash evaporation b) also apply mutatis mutandis to further preferred variants of the uses and the method as contemplated herein.

The inventive means, uses and methods and some of preferred embodiments thereof are summarized in the following points:
1. Cosmetic product comprising
a) a cosmetic preparation containing, relative to the total weight thereof,
   a1) from about 20 to about 99 wt % ethanol;
   a2) from about 0.05 to about 20 wt % fragrance;
b) a device for flash evaporation of cosmetic preparation a).
2. Cosmetic product according to item 1, wherein the flash evaporation device comprises a container b1) and a heater b2) and is designed such that
   the cosmetic preparation a) can be accommodated in the interior space of the container b1),
   the interior space of the container b1) at least partly filled with the cosmetic preparation a) is closable,
   the cosmetic preparation a) in the interior space of the container b1) can be heated by employing the heater b2), whereby pressure is increased,
   the heated cosmetic preparation a) can be relaxed out of the interior of container b1) into the surrounding atmosphere, whereby pressure is reduced.
3. Cosmetic product according to any one of the preceding items, wherein the proportion by weight of ethanol a1) to the total weight of cosmetic preparation a) is from about 30 to about 90 wt %, preferably from about 40 to about 80 wt %.
4. Cosmetic product according to any one of the preceding items, wherein the proportion by weight of water and ethanol to the total weight of the cosmetic preparation is at least about 60 wt %, preferably at least about 80 wt %, particularly preferably at least about 90 wt % and especially at least about 95 wt %.
5. Cosmetic product according to any one of the preceding items, wherein cosmetic preparation a) comprises ethanol and water and the weight ratio of ethanol to water is in the range from about 50:1 to about 1:2, preferably about 40:1 to about 1:1 and especially about 30:1 to about 10:1.
6. Cosmetic product according to any one of the preceding items, wherein the proportion by weight of fragrance to the total weight of the cosmetic preparation a) is from about 0.1 to about 15 wt %, and particularly from about 0.5 to about 10 wt %.
7. Cosmetic product according to any one of the preceding items, wherein fragrance a2) has a boiling point above 80° C., preferably above 120° C. and particularly above 160° C.
8. Cosmetic product according to any one of the preceding items, wherein cosmetic preparation a) contains from about 0.02 to about 4.0 wt %, preferably from about 0.05 to about 2.0 wt % and particularly from about 0.1 to about 1.0 wt % nonionic surfactant a3) relative to the total weight thereof.
9. Cosmetic product according to item 8, wherein nonionic surfactant a3) is selected from the group of PEG derivatives of hydrogenated castor oil, particularly preferably the group of PEG-40 Hydrogenated Castor Oil and PEG-60 Hydrogenated Castor Oil.
10. Cosmetic product according to any one of the preceding items, wherein cosmetic preparation a) contains a film-forming polymer a4) in a quantity of from about 0.1 to about 20 wt %, preferably from about 0.5 to about 15 wt % and particularly from about 1.0 to about 10 wt % relative to the total weight thereof.
11. Cosmetic product according to one of the preceding items, wherein at least about 80 wt %, preferably at least about 90 wt % and particularly at least about 97 wt % of cosmetic preparation a) relative to the total weight thereof consists of components a1) and a2).
12. Cosmetic product according to any one of the preceding items, wherein at least about 80 wt %, preferably at least about 90 wt % and particularly at least about 97 wt % of cosmetic preparation a) relative to the total weight thereof consists of components a1), a2) and component a3).
13. Use of a cosmetic composition a) comprising, relative to the total weight thereof, a1) from about 20 to about 99 wt % ethanol; a2) from about 0.05 to about 20 wt % fragrance as process materials in a flash evaporation device.
14. Use of a product according to any one of items 1 to 12 for applying a cosmetic preparation a) to human skin or human hair.
15. Use of a product according to any one of items 1 to 12 for fragrancing human skin or human hair.
16. Method for fragrancing human skin or human hair, in which a cosmetic composition a) comprising, relative to the total weight thereof
   a1) from about 20 to about 99 wt % ethanol;
   a2) from about 0.05 to about 20 wt % fragrance
      is applied to the hair or skin by employing a flash evaporation device.
17. Method of item 16, wherein
   a partial quantity of the cosmetic preparation a) present in a reservoir, inside which a pressure corresponding to ambient pressure prevails, is transferred from said reservoir to a container b1);
   the flow between reservoir and container b1) is then interrupted by a flow control component, such that the flow of cosmetic preparation a) from the reservoir into container b1) can be interrupted;
   then, the cosmetic preparation a) in container b1) which is closed off from the surrounding atmosphere is heated by a heater, so that the pressure inside container b1) increases to values above ambient pressure, preferably to values between about 1.1 and about 8 bar, particularly to values between about 1.2 and about 4 bar;
   then, the container b1) under a pressure above ambient pressure opens in such manner as to allow at least a partial quantity, preferably at least about 50 wt %, more preferably at least about 80 wt % and particularly at least about 90 wt % of the cosmetic preparation inside container b1) to escape from the container b1) into the environment, at the same time reducing the pressure that prevails in container b1) at the time container b1) is opened.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product comprising:
a) a cosmetic preparation comprising, relative to the total weight thereof,
a1) from about 20 to about 99 wt % ethanol, and
a2) from about 0.05 to about 20 wt % fragrance; and
b) a device for flash evaporation of cosmetic preparation a).

2. Cosmetic product according to claim 1, wherein the flash evaporation device comprises a container b1) and a heater b2) and is designed such that:
the cosmetic preparation a) can be accommodated in the interior space of the container b1),
the interior space of the container b1) at least partly filled with the cosmetic preparation a) is closable,
the cosmetic preparation a) in the interior space of the container b1) can be heated by employing the heater b2), whereby pressure is increased, and
the heated cosmetic preparation a) can be relaxed out of the interior of container b1) into the surrounding atmosphere, whereby pressure is reduced.

3. Cosmetic product according to claim 1, wherein the proportion by weight of ethanol a1) to the total weight of cosmetic preparation a) is from about 30 to about 90 wt %.

4. Cosmetic product according to claim 1, wherein the proportion by weight of fragrance to the total weight of the cosmetic preparation a) is from about 0.1 to about 15 wt %.

5. Cosmetic product according to claim 1, wherein fragrance a2) has a boiling point above about 80° C.

6. Cosmetic product according to claim 1, wherein cosmetic preparation a) comprises from about 0.02 to about 4.0 wt % of a nonionic surfactant a3) relative to the total weight thereof.

7. Cosmetic product according to claim 1, wherein at least about 80 wt % of cosmetic preparation a) relative to the total weight thereof comprises components a1) and a2).

8. A cosmetic composition a) comprising, relative to the total weight thereof,
a1) from about 20 to about 99 wt % ethanol, and
a2) from about 0.5 to about 20 wt % fragrance;
wherein the cosmetic composition a) is utilized as a material in a flash evaporation device.

9. Cosmetic product according to claim 1, wherein the cosmetic product is utilized for applying a cosmetic preparation a) to human skin or human hair.

10. Cosmetic product according to claim 1, wherein the cosmetic product is utilized for fragrancing human skin or human hair.

11. Method for fragrancing human skin or human hair, the method comprising applying a cosmetic composition a) to the hair or skin by employing a flash evaporation device, the cosmetic composition a) comprising, relative to the total weight thereof,
a1) from about 20 to about 99 wt % ethanol; and
a2) from about 0.05 to about 20 wt % fragrance.

12. Method according to claim 11, wherein
a partial quantity of the cosmetic preparation a) present in a reservoir, inside which a pressure corresponding to ambient pressure prevails, is transferred from the reservoir to a container b1);
the flow between reservoir and container b1) is then interrupted by a flow control component, such that the flow of cosmetic preparation a) from the reservoir into container b1) can be interrupted;
then, the cosmetic preparation a) in container b1) which is closed off from the surrounding atmosphere—is heated by a heater, so that the pressure inside container b1) increases to values above ambient pressure; and
then, the container b1) under a pressure above ambient pressure opens such that to allow at least a partial quantity of the cosmetic preparation inside container b1) to escape from the container b1) into the environment, at the same time reducing the pressure that prevails in container b1) at the time container b1) is opened.

13. Cosmetic product according to claim 6, wherein the nonionic surfactant a3) is selected from the group of PEG derivatives of hydrogenated castor oil.

14. Cosmetic product according to claim 13, wherein the nonionic surfactant a3) is selected from the group of PEG-40 Hydrogenated Castor Oil, PEG-60 Hydrogenated Castor Oil, and combinations thereof.

15. Cosmetic product according to claim 13, wherein at least about 80 wt % of cosmetic preparation a), relative to the total weight thereof, comprises of components a1), a2) and component a3).

16. Cosmetic product according to claim 1, wherein cosmetic preparation a) comprises ethanol and water and the weight ratio of ethanol to water is in the range from about 50:1 to about 1:2.

17. Cosmetic product according to claim 16, wherein the proportion by weight of water and ethanol to the total weight of the cosmetic preparation a) is at least about 60 wt %.

18. Cosmetic product according to claim 1, wherein the proportion by weight of ethanol a1) to the total weight of cosmetic preparation a) is from about 40 to about 80 wt %.

19. Cosmetic product according to claim 5, wherein fragrance a2) has a boiling point above about 120° C.

20. Cosmetic product according to claim 19, wherein fragrance a2) has a boiling point above about 160° C.
